(19) United States
(12) Patent Application Publication
(10) Pub. No.: US 2022/0015937 A1
(43) Pub. Date: Jan. 20, 2022

(54) PHALANGEAL BRACE AND COLD PACK

(71) Applicant: Carlos Gaston, Seattle, WA (US)

(72) Inventor: Carlos Gaston, Seattle, WA (US)

(21) Appl. No.: 16/931,349

(22) Filed: Jul. 16, 2020

(51) Int. Cl. A61F 5/01; A61F 7/02

(52) U.S. Cl. CPC A61F 5/0118; A61F 2007/0037; A61F 7/02

(57) ABSTRACT

A finger brace may provide flexion and/or lateral support for an injured finger. The finger brace may restrict motion of an injured finger and simultaneously provide a cold compress to an injured finger. The cold compress may be selectively attachable to the finger brace and provide localized treatment to the injured finger. A strap may secure the device onto one or more fingers of a user. The finger brace provides multi-modal treatment to an injured finger.

### BACKGROUND

The field of the present disclosure is related to the first-aid treatment of tissue, especially that of the fingers and hand.

One of the most common hand injuries when playing ball sports (e.g., basketball, volleyball, football, and others) is a jammed finger. The injury often occurs from the blunt impact or forced motion to the distal or proximal interphalangeal joint of the fingers. A jammed finger often impacts the collateral ligaments associated with one of these joints and is typically a sprained joint. The sprained joint may include a stretching of the collateral ligaments, a partial tear of one or more collateral ligaments, or a complete tear of one or more collateral ligaments.

Typical symptoms of a sprained joint are pain, swelling, redness, and tenderness, and a reduced range of motion. In some cases, an avulsion fracture may accompany a sprain injury.

One common treatment for a sprained finger is to apply ice to the finger. This typically involves applying a bulky ice pack to the finger. The ice pack may be a previously frozen gel pack, or may be as simple as a bag containing ice that is put on the injured finger. The ice pack typically contacts the injured finger along with one or more uninjured fingers, and other parts of the hand. In some cases, a jammed finger may be taped to an adjacent finger to help limit motion of the injured finger.

However, prior methods and apparatuses of treating finger injuries can result in less than ideal ice application, as typical treatments inadvertently apply ice to uninjured parts of the hand and adjacent fingers. In addition, splinting a finger by a mechanical split or by “buddy taping” it to an adjacent finger reduce the efficacy of any applied cold compress to the injury.

While these treatments have provided benefit for many athletes, it would be desirable to provide improvements that result in more accurate and targeted cold therapy application, more robust or easily implemental system to treat such injuries, and other features that will become apparent from the following description.

### SUMMARY

Embodiments that follow provide several advantages to the treatment of an injured finger, such as caused by jamming a finger. Notably, the described device provides a brace or splint to restrict movement of the finger. The brace may restrict bending movement of the finger and additionally provide lateral support. The device also provides a localized cold compress to the injured area. The cold compress relieves pain and swelling, thus helping the injury to heal faster.

According to some embodiments a finger brace comprises a brace member having a generally flat surface for supporting the palmar surface of one or more fingers; an upper member coupled to the brace member; a fastening member configured to secure the brace member and the upper member to the one or more fingers; and a cold pack carried by the upper member.

In some embodiments, the brace member is formed of a substantially rigid material and configured to restrict movement of the one or more fingers at one or more of the MCP joint, the proximal IP joint, and the distal IP joint.

In some instances, the fastening member is formed as a strap configured to wrap around the brace member and the upper member. The fastening member may have a first end affixed to the brace member or the upper member, and a second end configured to selectively attach to a portion of the fastening member. In some cases, the first end is configured with a first component of a hook and loop fastener and the portion of the fastening member is configured with a second component of the hook and loop fastener. That is, the first end may have either a hook or a loop portion and the second component has the other portion of the hook and loop fastening system.

The fastening member may be formed of an elastic material that can stretch and provide adjustability of the finger brace onto the one or more fingers.

In some embodiments, the cold pack is selectively attachable to the upper member. For instance, selective attachment can be by hook and loop fastener, by a groove and a protrusion, or some other cooperating structure.

In some embodiments, the upper member defines an internal cavity and the cold pack is located within the internal cavity.

In some cases, a cushion is provided and is positioned adjacent the brace member, adjacent the upper member, or both. For instance, the cushion may be a textile material, an inflatable material, a synthetic material, or some other material. In some instances, the cushion provides a boundary layer between the cold pack and the one or more fingers. That is, the cushion is located between an injured finger and the cold pack.

According to some embodiments, a finger brace includes a brace member having a supporting surface for supporting the palmar surface of one or more fingers; an upper support coupled to the brace member; a strap to secure the brace member and the upper member to the one or more fingers; and a cold pack carried by the upper member.

In some cases, the upper member comprises the cold pack. In some examples, the cold pack is selectively attachable to the finger brace.

The upper member may define an internal cavity, and the cold pack is located within the internal cavity. In such cases, the entire finger brace may be placed into a freezer to reduce the temperature of the cold pack.

In some embodiments, the cold pack is fillable with a heat-absorbing material.

The strap may be configured to extend around one or more fingers including an uninjured finger. For example, where a pointer finger is injured, the brace member may provide support to the pointer finger, while the strap wraps around one or more of the pointer finger, middle finger, ring finger, or pinky finger.

In some embodiments, the strap is selectively attachable to secure the one or more fingers. The strap may include any suitable structure to allow at least one end of it to be removably attached, such as snaps, hook and loop, a buckle, adhesive, a clip, or other suitable structure.

In some instances, the supporting surface of the finger brace extends to a location between a distal IP joint and a fingertip.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1A shows a phalangeal brace and cold pack brace are shown from a top view for supporting a plurality of fingers, in accordance with some embodiments;

FIG. 1B illustrates a phalangeal brace and cold pack from a rear isometric view; in accordance with some embodiments;

FIG. 1C illustrates a phalangeal brace and cold pack from a side isometric view, in accordance with some embodiments;

FIG. 2A illustrates a phalangeal brace and cold pack from a perspective view for treating a single finger, in accordance with some embodiments;

FIG. 2B illustrates the phalangeal brace and cold pack of from a rear perspective view, in accordance with some embodiments;

FIG. 2C illustrates a phalangeal brace and cold pack from a side view, in accordance with some embodiments;

FIG. 3A illustrates a thumb brace and cold pack from a front perspective view, in accordance with some embodiments;

FIG. 3B illustrates a thumb brace and cold pack from a side view, in accordance with some embodiments; and

FIG. 3C illustrates a thumb brace and cold pack from a rear perspective view, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

In many cases, a jammed or sprained finger will benefit from the application of a cold compress, such as ice, a cold pack, or some other suitable method of applying a reduced temperature device to an injured joint. For purposes of this disclosure, a cold pack will refer to any suitable application of a material providing a reduced temperature below ambient and may include any suitable material such as, without limitation, ice, gel, metal, water, or other liquid, solid, or phase change material.

In addition to a cold pack, a jammed (e.g., sprained) finger may benefit from a splint or other device to temporarily reduce mobility of the injured joint. According to some embodiments, a brace and cold pack is provided that is capable of both reducing the mobility of the joint and applying a localized cold compress to the affected area. Embodiments as described herein are particularly suitable for jammed fingers, as some of the embodiments described herein will immobilize only the affected joint and apply a cold pack directly to the affected area, while reducing or limiting the application of cold temperatures to uninjured parts of the hand.

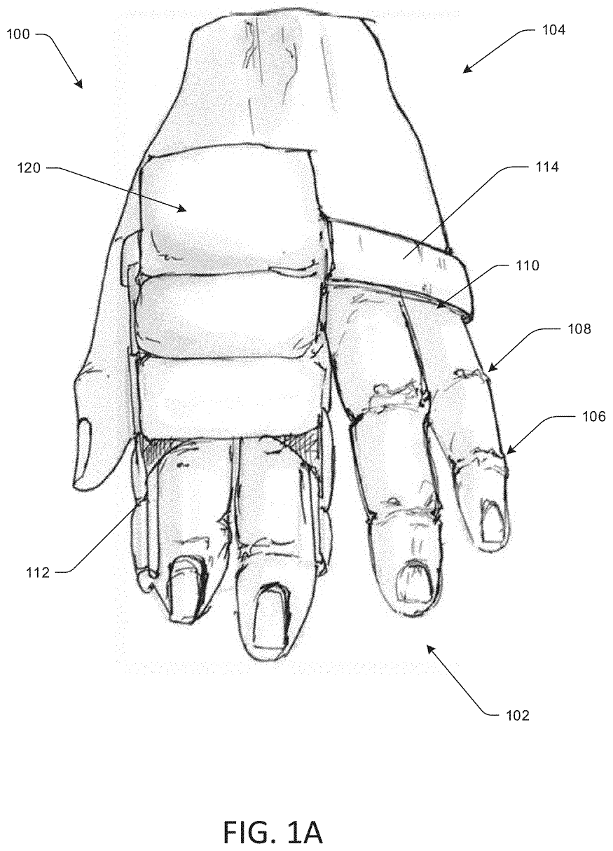

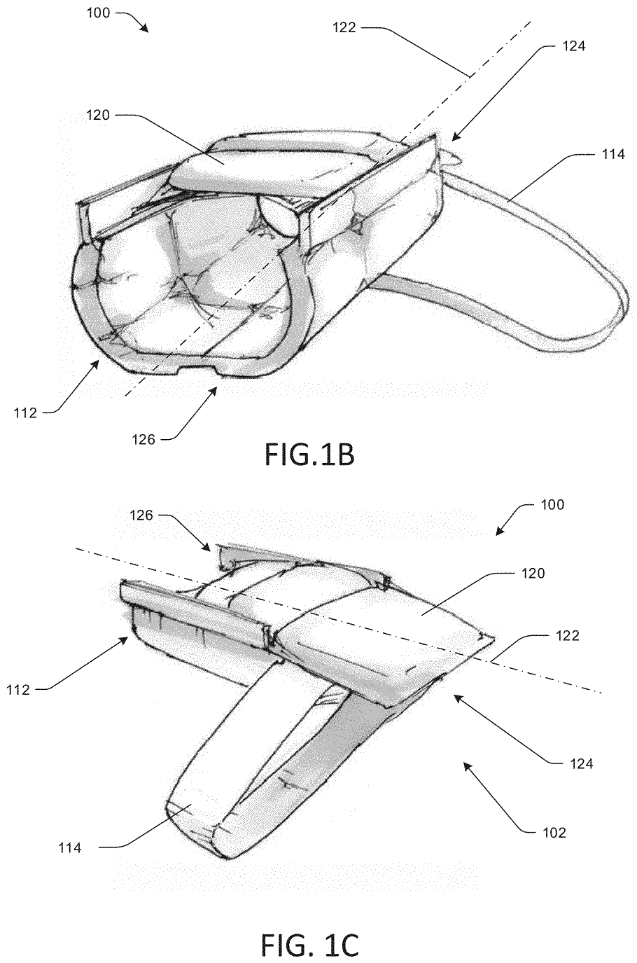

With reference to FIG. 1A, a brace and cold pack 100 is illustrated as applied to a plurality of fingers 102 of a hand 104.

For purposes of this disclosure, a hand 104 has five digits, the thumb (or the first digit), an index finger (or second digit), a middle finger (or third digit), a ring finger (or fourth digit), and a little finger (or fifth digit). When referring to the four fingers of a user, this describes the second, third, fourth, and fifth digits, exclusive of the thumb. The four fingers of a user will typically each have a plurality of interphalangeal joints (IP joints). Specifically, each of the four fingers will have a distal interphalangeal joint 106, a proximal interphalangeal joint 108, and a metacarpophalangeal joint (“MCP” joint) 110 (e.g., the knuckle joint).

A jammed finger results when a blunt impact pushes the tip of the finger toward the hand. The impact force can stretch, or tear, the ligaments of the finger which causes a sprain. Accordingly, the injuries referred to as a “jammed finger” or a “sprained finger” are used interchangeably throughout. If the blunt force exceeds a threshold force, it can cause tendon damage or a bone fracture. A jammed finger will typically swell and be painful and should be treated. Whether the sprain is mild or severe, the first aid to the finger may be the same in many cases.

A brace and cold pack 100 provides both a splint and a cold pack, according to some embodiments. The device may include a brace member 112 that supports the jammed finger or fingers and inhibits movement of the fingers, or at least reduces movement of the fingers. The brace member 112 may extend along one surface of the finger, such as the palmar surface to reduce the ability of the injured finger to bend at one or more of the distal IP joint, the proximal IP joint, or the MCP joint. In some embodiments, the brace member 112 surrounds one or more sides of the finger to provide lateral support to the finger or reduce lateral movement of the finger.

In some instances, the brace member is coupled to a fastening member 114 that is configured to secure the brace member 112 to the fingers 102. The fastening member 114 may be any suitable member that holds the brace member adjacent to one or more fingers. In some instances, the fastening member is formed of any suitable material, and may be formed of a textile material, a stretchy material, a plastic material, or a combination of materials. In some embodiments, the fastening member 114 is formed of one or more of cloth, elastic, leather, spandex, natural or synthetic materials, or a combination of materials.

One or more cold packs 120 may be provided to apply a cold compress to the injured finger. In some embodiments, one, two, three, four or more cold packs 120 may be carried by, affixed to, are secured to the brace member 112, as will be described hereinafter.

In some embodiments, the brace member 112 reduces flexion of one or more of the distal IP joint 106, proximal IP joint 108, or MCP joint 110 and does not inhibit other finger movements, such as, for example, one or more of extension (moving the base of the fingers away from the palm), adduction (moving the fingers toward the middle finger), or abduction (spreading the fingers away from the middle finger).

With reference to FIGS. 1B and 1C, a brace and cold pack 100 is shown from a rear and side perspective view, respectively. The brace member 112 may be rigid, or semi-rigid, and provide support to one or more fingers. In some cases, the brace member 112 is formed of any suitable metal or polymer, and may define an internal chamber that may be filled with a suitable cold pack material. In some cases, the entire brace and cold pack 100 can be cooled, such as within a freezer, and a cold pack material within the brace can be cooled to provide cold therapy to the palmar side of the finger. In some embodiments, the brace member 112 is formed of a material that may be plastically deformed by hand to facilitate shaping the brace member 112 to be adapted to one or more fingers.

In some cases, a cold pack may be removably attached to a dorsal side of the brace member 112. For example, a cold pack may be attached to a dorsal side of the brace member 112 and apply cold therapy to the dorsal side of one or more fingers. For clarity, the dorsal side of the finger corresponds with the back of the hand.

One or more cold packs 120 can be secured to the brace member 112, such as by clips, loops, hook and loop, channels, adhesive, straps, fitting in a sleeve, stitching, or some other suitable structure or a combination of structures. In some cases, the one or more cold packs 120 are attached to the brace member 112 by the fastening member 114. For example, the fastening member 114 may wrap around a finger and around the brace member 112. Where a cold pack 120 is placed on the dorsal surface of the brace member 112, the fastening member 114 can wrap around the cold pack 120 and secure it to the brace member 112.

The fastening member 114 may be adjustable in length, such as to allow a suitably tight fit to many different finger sizes. For example, one end of the fastening member 114 may be fixedly attached to a portion of the brace member 112 and a free end of the fastening member can be manipulated to secure the fastening member 114. In some embodiments, the fastening member comprises hook and loop fastener, such that the fastening member 114 can wrap around the finger, the brace member 112, or both, and a loop portion of the fastening member 114 can be secured to a hook portion of the fastening member. The hook and loop fastening system allows the fastening member 114 to be selectively adjustable in length to provide a custom fit and for a variable tightness on the finger. According to some embodiments, the hook and loop fastening system forms a part of the fastening member 114, such that both the hook and the loop portions are carried by the fastening member 114. In some cases, one of the hook or the loop portions is carried by the fastening member 114 and the other one of the hook or the loop portions is carried by the brace member 112.

The brace member 112 has an elongate axis 122 that generally follows the direction of the fingers, when worn. In some embodiments, the brace member 112 is substantially flat in the direction of the elongate axis 122. In some instances, the brace member is curved having a constant or changing radius about the elongate axis 122. The brace member 114 has a distal end 124 and a proximal end 126. In some embodiments, the fastening member 114 is closer to the distal end 124 than the proximal end 126. In some cases, more than one fastening member 114 is provided, such as a first fastening member closer to the distal end 124 and a second fastening member closer to the proximal end. In some embodiments, the fastening member may be tubular with an axis of the tubular member extending generally parallel with the elongate axis 122 of the brace member 112.

One or more cold packs 120 may be carried by the brace member 112, or may be formed integrally therewith. For example, a removable cold pack 120 can be selectively attached to the brace member through any suitable structure. In some cases, the removably attachment is provided by bands, clips, hook and loop, magnets, a channel, a snap, an elastic, or may be held in place by the fastening member 114. In some embodiments, one or more of the cold packs 120 are permanently affixed to the brace member 112, and in some cases, one or more of the cold packs 120 may be internal to the brace member 112, such as by filling an internal cavity of the brace member 112 with a suitable cold pack material.

The material in the one or more cold packs 120 can be any suitable material. The cold pack material may include any material, and in some cases, is water, refrigerant gel, liquid, or any other material that can absorb a considerable amount of heat. In some cases, the cold packs may be filled with different materials. For example, a cold pack 120 internal to the brace member 112 may be filled with a material that remains a slow-flowing gel, even at temperatures of 0° C., −10° C., −20° C., or lower, and an exterior cold pack 120 may be filled with water, ice, or another liquid, gel, solid, or any other suitable material. In some cases, the cold packs may be filled with the same material. By way of example, suitable materials that remain a slow-flowing gel at −20° C. or less include hydroxyethyl cellulose, sodium polyacrylate, or silica gel.

One or more of the cold packs 120 may alternatively be an instant cold pack, in which two materials can be selectively mixed to create an endothermic chemical reaction. For example, one or more ice packs may include water and a chemical such as ammonium nitrate, calcium ammonium nitrate, or urea. Upon use, a user may squeeze the cold pack 120 to rupture a container holding the water and allow the water to mix with the solid to dissolve the solid in an endothermic reaction which quickly absorbs heat from the surroundings thereby lowering the temperature of the cold pack.

The cold packs 120, the brace member 112, or both may be covered with a suitable covering to inhibit direct contact between a user and the cold pack for an extended period of time. As an example, the brace member may be covered with a suitable covering, such as cotton, latex, polyethylene, polyvinylchloride, a natural or synthetic material or some other suitable material or combination of materials. The covering may be provided as a permanent cover, or as a replaceable covering for the brace member 112 and may be removable to allow the covering to be washed, for instance. The covering may be tubular and allow a user to insert one or more injured fingers into the tubular covering.

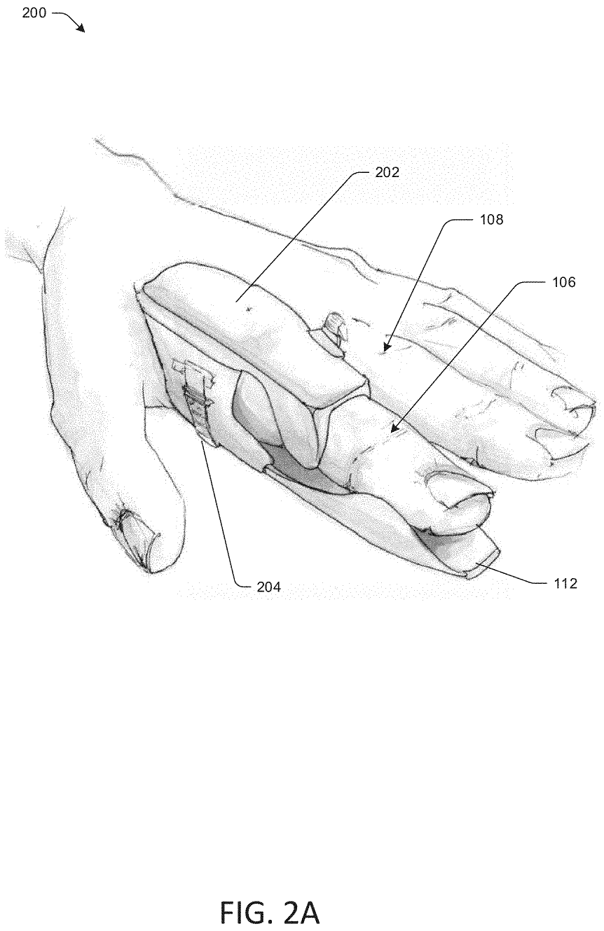

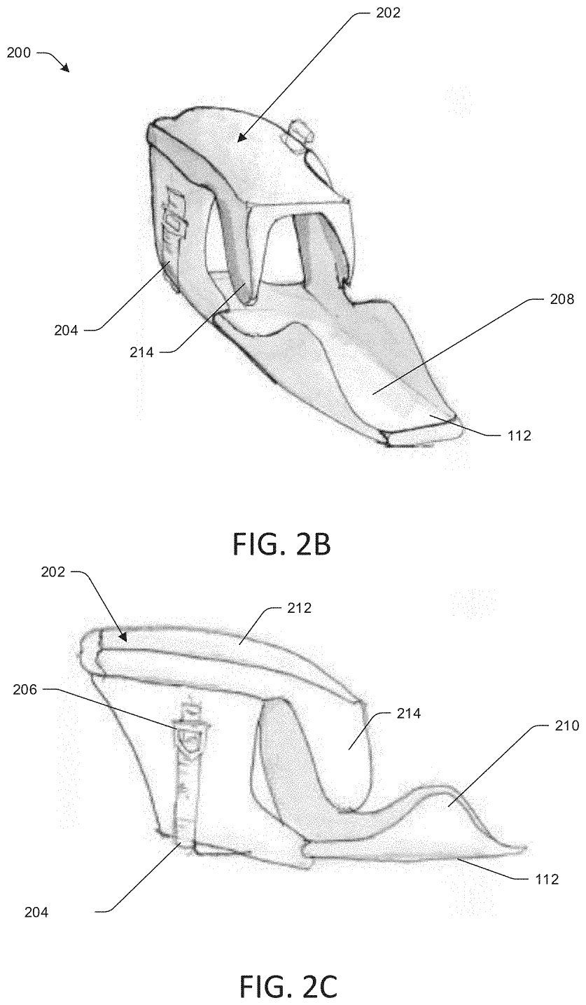

FIGS. 2A, 2B, and 2C illustrate a brace and cold pack 200 configured to support a single digit. The brace and cold pack 200 may be similar to other embodiments in many respects, such as the brace and cold pack 100 of FIG. 1A. One of the primary differences with the embodiment shown in FIG. 2A is the single-digit configuration. The brace and cold packs may otherwise share many, or all, of the same features, properties, and construction.

In some cases, the brace and cold pack 200 may include an upper member 202 that may be used to secure the brace member 112 to the finger. Alternatively, or in addition, the upper member 202 may secure one or more cold packs to the finger. In some cases, the upper member 202 is hingedly connected to the brace member 112 and can be selectively opened, such as to receive a finger, or closed to secure the brace member 112 to the finger. The upper member 202 may include a cold pack, such that when the upper member 202 is moved toward the brace member 112, the upper member 202 applies a cold pack to the finger within the brace and cold pack 200. The upper member may be rigid, flexible, or semi flexible. In some cases, the upper member is articulated to allow the upper member to contour to engage with the finger in the brace and cold pack 200. The upper member 202, the brace member 112, or both, may have a covering to separate the finger of a user from direct contact with the cold pack, the brace member 112, or the upper member 202.

A proximal fastening member 204 may be provided to allow the brace and cold pack 200 to be secured, and tightened to a desired degree to the finger. The proximal fastening member 204 may be any suitable fastening and/or tightening mechanism, such as those previously described in relation to FIGS. 1A-1C.

The brace member 112 may extend commensurate with, or beyond, the distal end of the finger to provide support along the entire palmar side of the injured finger. In some cases, the brace member 112 may be shorter than the finger, such as to allow limited movement of one or more of the distal IP joint 106 or the proximal IP joint. As described before, the brace member 112 may be formed of a material that provides for plastic deformation by hand to accommodate any size or shape of finger. However, in some cases, the brace member 112 is formed of a material that does not allow for plastic deformation by hand.

Optionally, the brace and cold pack 200 may include a proximal fastening member 204 and a distal fastening member that are configured to wrap around the brace and cold pack 200 at more than one location along the elongate axis. However, in some cases, a fastening member 204 may be sufficiently wide that it extends halfway, or more, along the elongate axis of the brace member 112.

The fastening member 204 may comprise a strap. In some cases, the fastening member 204 has a first end that is securely coupled to the brace member 112. The fastening member 204 may have a second end that is uncoupled from the brace member 112. The second end may be selectively coupled to the brace member 112 or a portion of the fastening member 204 through any suitable releasable coupling structure. In some cases, the brace member 112, or the upper member 202, may include a D-ring 206 or some suitable structure that allows the second end of the fastening member to pass therethrough and be cinched to a desired tightness. The second end of the fastening member 204 may carry one of a hook or a loop fastening system and be configured to cooperate with the other portion of a hook and loop fastening system to selectively cinch and secure the fastening member 204 onto one or more fingers. In some embodiments, the fastening member 204 is fixed to the brace member 112 at both ends, and is formed of an elastic material to allow the brace member 112 to slide over an injured finger and the elasticity of the fastening member 204 keeps the brace member 112 in place.

The brace member 112 can be formed of any suitable shape. In some examples, the brace member 112 has a generally flat portion 208 for supporting the palmar side of an injured finger. The brace member 112 may optionally include one or more raised side sections 210 for providing lateral support to the injured finger.

The upper member 202 may have a generally concave surface 212 configured to follow the contour of an MCP joint of a finger. In some embodiments, the upper member 202 includes one or more side supports 214 to provide lateral support to the MCP joint.

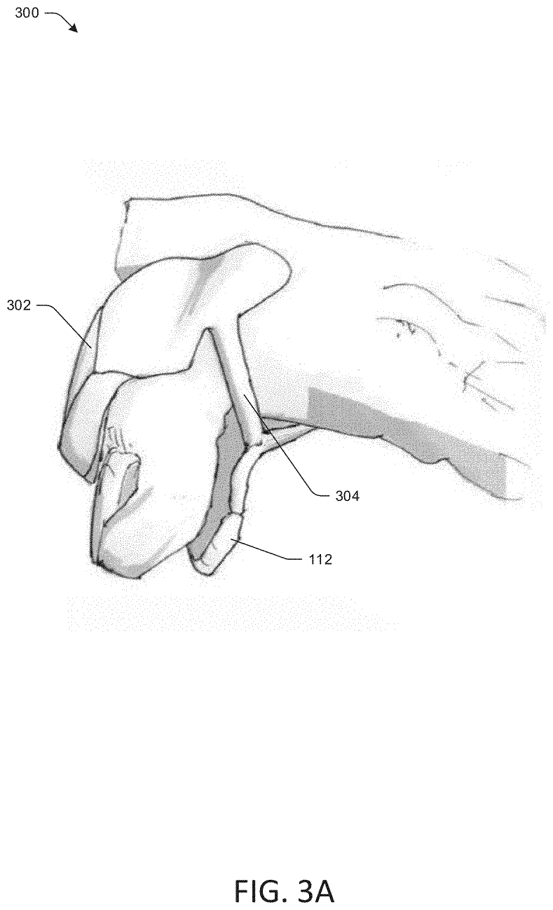

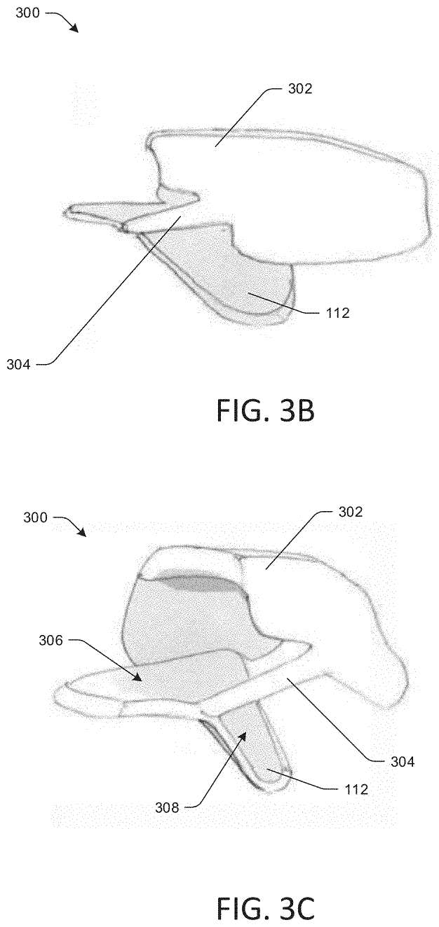

FIG. 3A illustrates an example of a thumb brace and cold pack 300 from a front perspective view. A brace member 112 provides support to the thumb on the palmar side of the thumb. In some embodiments, the brace member 112 supports the MCP joint and the IP joint. The brace member 112 may be as substantially described herein, that is, it may provide rigid or semi-rigid support to the thumb to inhibit, or at least reduce, movement of one or more of the MCP joint and the IP joint.

An upper member 302 supports the dorsal side of the MCP joint and restricts movement of the thumb. The upper member 302 may carry one or more cold packs, as described herein, which may be detachable from the upper member 302, and in some embodiments, are permanently affixed to, or within, the upper member 302. The cold packs may be any suitable cold packs, such as ones filled with water, ice, liquid, gel, or chemicals providing an endothermic chemical reaction when mixed.

A spacer 304 connects the brace member 112 to the upper member 302. The space created by the spacer 304 provides an aperture for an injured thumb to be inserted between the brace member 112 and the upper member 302. The spacer 304 is configured to be in close proximity to the hand at a location between the thumb and forefinger when worn by a user.

The brace member 112, upper member 302, and spacer 304 may be formed of a rigid material to provide support to a thumb and restrict movement thereof. Suitable materials include any of a variety of metals, polymers, composites, or any combination of suitable materials. In some embodiments, a cover is affixed to the brace and cold pack 300 to provide a separation between the users thumb and the cold pack. The cover may be permanently affixed or selectively removable. In some cases, the cover is a sock configured to receive a thumb, thereby covering the thumb and protecting it from the temperature of the cold pack, and configured to wick away sweat from the injured digit.

FIGS. 3B and 3C illustrate an example of a thumb brace and cold pack 300 from a side view and rear perspective view. A brace member 112 is configured to provide support to an injured thumb when worn by a user. The brace member includes an MCP support 306 and an interproximal support 308. The MCP support 306 and the interproximal support 308 each define generally planar support surfaces that are oriented at an angle with respect to one another to fit the anatomical geometry of the hand at the MCP joint.

In some embodiments, the brace member 112 is fixed to the upper member 302 with a fixed orientation, that is, the upper member 302 does not move relative to the brace member 112. In some embodiments, the brace member 112 is coupled to the upper member 302 by a pliable coupling, which may include a hinge, a pliable member, a pin, or some other structure that allows a degree of movement between the brace member 112 and the upper member 302. Such pliable couple may allow the brace and cold pack 300 to selectively tightened onto the thumb of a user.

In use, a user inserts a thumb into the device between the brace member 112 and the upper member 302 with the spacer 304 between the thumb and forefinger. In some cases, a fastening member (not shown), which may be similar to the fastening members described in previous embodiments herein, may be used to secure and/or tighten the brace and cold pack 300 onto the thumb of the user. In some embodiments, one or more cold packs are permanently attached to the brace and cold pack 300 and the brace and cold pack 300 may be stored in a freezer until ready for use. In some cases, a cold pack is a separate device that is selectively attached to the brace member 112, the upper member 302, or both. The cold pack may be attached through any suitable attachment mechanism or structure, and in some cases, is attached through hook and loop fastener, straps, elastics, cooperating structures such as snaps, grooves and protrusions, clips, locks, or another structure or combination of structures.

The brace and cold pack 300 provides multiple forms of treatment for an injured thumb, such as restricting motion of the MCP joint and/or the IP joint, while simultaneously applying a cold compress to the affected location. The cold compress helps reduce swelling and associated pains.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

As used herein, the term “or” is used inclusively to refer items in the alternative and in combination.

As used herein, characters such as numerals refer to like elements.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

What is claimed is:

1. A finger brace, comprising:
a brace member having a generally flat surface for supporting the palmar surface of one or more fingers;
an upper member coupled to the brace member;
a fastening member configured to secure the brace member and the upper member to the one or more fingers; and
a cold pack carried by the upper member.

2. The finger brace as in claim 1, wherein the brace member is formed of a substantially rigid material and configured to restrict movement of the one or more fingers at one or more of an MCP joint, a proximal IP joint, and a distal IP joint.

3. The finger brace as in claim 1, wherein the fastening member comprises a strap configured to wrap around the brace member and the upper member.

4. The finger brace as in claim 3 wherein the fastening member has a first end affixed to the brace member or the upper member, and a second end configured to selectively attach to a portion of the fastening member.

5. The finger brace as in claim 4, wherein the first end is configured with a first component of a hook and loop fastener and the portion of the fastening member is configured with a second component of the hook and loop fastener.

6. The finger brace as in claim 3, wherein the fastening member comprises an elastic material configured to stretch and provide adjustability of the finger brace onto the one or more fingers.

7. The finger brace as in claim 1, wherein the cold pack is configured for selective attachment to the upper member.

8. The finger brace as in claim 7, wherein the selective attachment is by hook and loop fastener.

9. The finger brace as in claim 7, wherein the selective attachment is by a groove and a protrusion.

10. The finger brace as in claim 1, wherein the upper member defines an internal cavity and the cold pack is located within the internal cavity.

11. The finger brace as in claim 1, further comprising a cushion positioned adjacent the brace member, adjacent the upper member, or both.

12. The finger brace as in claim 11, wherein the cushion provides a boundary layer between the cold pack and the one or more fingers.

13. A finger brace, comprising:
a brace member having a supporting surface for supporting the palmar surface of one or more fingers;
an upper support coupled to the brace member;
a strap to secure the brace member and the upper support to the one or more fingers; and
a cold pack carried by the upper support.

14. The finger brace as in claim 13, wherein the upper support comprises the cold pack.

15. The finger brace as in claim 13, wherein the cold pack is selectively attachable to the finger brace.

16. The finger brace as in claim 13, wherein the upper support comprises an internal cavity, and the cold pack is located within the internal cavity.

17. The finger brace as in claim 13, wherein the cold pack is fillable with a heat-absorbing material.

18. The finger brace as in claim 13, wherein the strap is configured to extend around one or more fingers including an uninjured finger.

19. The finger brace as in claim 13, wherein the strap is selectively attachable to secure the one or more fingers.

20. A finger brace comprising:
a finger support configured to support an MCP joint and a proximal IP joint on a palmar side of a finger; and
an ice pack carried by the finger support configured to apply a cold compress to the dorsal side of the MCP joint and the proximal IP joint.
